(19) United States
(12) Patent Application Publication
(10) Pub. No.: US 2004/0260359 A1
(43) Pub. Date: Dec. 23, 2004

(54) Method for treating neurogenic illnesses responding to a low amperage electrical shock

(76) Inventor: Vincent Osrud, Vestal, NY (US)

Correspondence Address: FOX ROTHSCHILD O'BRIEN & FRANKEL LLP, PRINCETON PIKE CORPORATE CENTER, 997 LENOX DRIVE, BUILDING 3, LAWRENCEVILLE, NJ, 08648, US

(21) Appl. No.: 10/822,089

(22) Filed: Apr. 8, 2004

Related U.S. Application Data
(63) Continuation-in-part of application No. 10/230,447, filed on Aug. 30, 2002.

(51) Int. Cl. A61N 1/18

(52) U.S. Cl. 607/48

(57) ABSTRACT

The invention comprises a method and apparatus for treating neurogenic conditions and ailments with a specific type of electrical stimulation, whose intensity is adjusted to individual sensitivity and the particular nerve site, and then applied to the neurogenic site.

### RELATED PATENT APPLICATION

This application is a continuation-in-part of the previously filed U.S. patent application Ser. No. 10/230,447, filed: Aug. 30, 2002, entitled: Nerve Stimulator, Attorney Docket H-535; the teachings of which, are meant to be incorporated herein by way of reference.

### FIELD OF THE INVENTION

The present invention relates to methods for treating illness using electrical stimulation, and more particularly to a method of treating neurogenic illnesses responding to a low Amperage electrical shock.

### BACKGROUND OF THE INVENTION

The aforementioned United States Patent application described a handheld electrical stimulator used essentially for treating CFID Syndrome, fibromyalgia, myositis, Chronic Fatique Syndrome, and pain. The electrical stimulator of the prior invention was distinguished from the well-known, prior art, T.E.N.S. machine in many different characteristics, amongst which was its use of square wave potential instead of the saw-tooth, variable waveform used by the T.E.N.S. machine.

In general, the whole nature of pain and disease related to the nervous system (neurogenesis) has not been well understood, nor has electrical stimulation to treat medical problems.

The aforementioned patent application had described a method of treating CFIDS, fibromyalgia, associated ailments, and pain.

The current invention teaches new methods of treatment for diverse illnesses, and briefly explores the underlying benefits and rational of applying square wave potential to diseased areas of the human body. This invention has also redesigned the handheld unit by providing fixed electrodes. The fixed electrodes make it easier to apply the electrical stimulus to various parts of the body.

### SUMMARY OF THE INVENTION

In accordance with the present invention, a method and apparatus for treating diverse neurogenic illnesses, is disclosed. The action potential of the nerves in the sympathetic nervous system can be categorized into relationships of high and low action potential to the threshold of activation. For conditions like a fibrillating heart, the action potential is too high, or the threshold of the nerve firing is too low, and for conditions like post stroke victims, the action potential is too low, or the threshold of the nerve firing is too high.

The present treatment method comprises the technique of changing the action potential or threshold level of the nerves associated with a disease or condition, by mildly shocking these nerves. This allows the affected nerves to restart a normal nerve transmission, i.e. a transmission at a preferred, or normal pace. Applying electric stimulation will not always work due to necrosis, or death of treated tissue or nerves. Although the use of electrical stimulation is a common technique in treating many ailments, the method of this invention is very specific and unique. The apparatus possesses the ability to vary or adjust the current of the pulses to the needs of the individual. In order to work properly, the patient needs to feel the stimulus. The parts of the body have different stimulus sensitivities. For example, when applying the stimulating current to the head of a patient; most patients will feel the stimulus at a 1-milliampere level. Applying the stimulus to the back of the same individual, the milliamps would likely have to be increased towards the upper amperage range of about 33 milliamps. Individuals, themselves, vary in sensitivity, as well as the different sensitivity levels associated with the different parts of the body. Placement of the electrodes also depends on the condition being treated. For example, for a stroke patient, or a patient with nerve damage, carpal tunnel syndrome, and victims of assault (low action potential), the stimulation site is the large nerve plexus. For high action potential conditions and diseases, such as fibromyalgia, psoriasis, etc., the action sites are local, and comprise trigger point sites.

Electrical stimulation comprises the application of a pulsed, or periodic electrical voltage in the output range of approximately between 240 and 440 volts, at an approximate preferred current range of between 18 and 30 milliamps (the full range being approximately 1 to 33 milliamps). The shape of the periodic wave comprises a substantially square wave operating at approximately two pulses per second. The method of the invention applies this stimulation to the spine, muscles, and facial connections, also known as “trigger points”, for some conditions, and the large nerve plexus for other ailments.

At the present time, the method of this invention has successfully treated patients having the following conditions: fibromyalgia, chronic fatigue syndrome, temporo-mandibular joint syndrome, paresthesia, numbness, carpal tunnel syndrome, chronic pain (including migraine and general headaches), tennis elbow, tendonitis, arthritis, sciatica, bursitis, reflex sympathetic dystrophy, psoriasis, scleroderma, lupus erythematosus, post-stroke victims, Raynaud's disease, post-surgical numbness, and symptoms of multiple sclerosis.

It is an object of the present invention to provide an improved treatment for neurogenic ailments.

It is another object of this invention to provide an improved apparatus for electrically stimulating parts of the body in the treatment of neurogenic ailments, and pain.

### BRIEF DESCRIPTION OF THE DRAWINGS

A complete understanding of the present invention may be obtained by reference to the accompanying drawings, when considered in conjunction with the subsequent detailed description, in which:

FIG. 1 illustrates a schematic view of the improved electrical stimulator of this invention; and

FIG. 2 depicts a schematic view of an electrical circuit for providing pulsed square waveforms for the stimulator shown in FIG. 1.

### DESCRIPTION OF THE PREFERRED EMBODIMENT

Generally speaking, there are many neurogenic conditions that respond to mild and low amperage level electrical pulses. The invention comprises a method and apparatus for treating these conditions with a specific type of electrical stimulation, whose intensity is adjusted to the individual, the particular condition or disease being treated, and to the particular location responsive to the applied electrical stimulus.

Much of what we know about the operation of neurons comes from experiments conducted upon the giant axon of the squid. This giant axon extends from the head-to-tail of the squid, and is about 1 mm in diameter.

Neurons send messages electrochemically. Ions, the charged chemicals that surround the membrane of the nerve cells, may have a positive or negative charge. Some ions pass through the membrane, altering the state of the neuron. There are selective ion channels, and a pump that uses energy to move three sodium ions out of the neuron for every two potassium ions that are put into the neuron. Forces tend to balance out, and the neuron is said to have a resting potential. The mean resting potential is about 70 mV on both sides of the cell wall. This means that the inside of the neuron is 70 millivolts less than the outside. At rest, there are relatively more sodium ions outside the neuron vis-a-vis the inside potassium ions.

An action potential is when a neuron sends a signal down the axon, away from the cell body. Scientists some times refer to this as a “spike”, or “impulse”. The action potential is an explosion of electrical activity created by a depolarizing current. This means, that some stimulus causes the resting potential to move toward 0 mV. The neuron will fire an action potential, when the threshold level of −55 mV., is reached. The neuron will not fire until the threshold is reached. All action potentials are the same size in one nerve cell. Therefore, the neuron either reaches the threshold and fires, or it does not reach it, and does not fire. Outside, positively charged, sodium ions flowing across the neuron membrane, depolarize the neuron, and advance towards the threshold. Potassium channels take longer to open, and reverse the depolarization, so that the neuron can return to the resting potential of −70 mV.

As aforementioned, the method and apparatus of this invention applies pulsed, or periodic electrical voltage in the output range of approximately between 240 and 440 volts, at an approximate, preferred current range of between 18 and 30 milliamps, from an overall approximate operative current range of 1 to 33 milliamps. The shape of the periodic wave comprises a substantially square wave operating at approximately two pulses per second. The method and apparatus of the invention applies this stimulation to the spine, muscles, and facial connections, also known as “trigger points” in some conditions, and to the large nerve plexus for other ailments.

Similar stimulation can be obtained from a nerve stimulator, called: The Sparkie Nerve Stimulator, Model No. 54130R, sold by Dupaco, Inc., Oceanside, Calif., operating in a “twitching mode”. This stimulator, however, is incapable of adjusting the electrical signals to provide for a change of current, nor does the prior art teach where and how to apply this type of electrical signal. This becomes a serious drawback, for example, in the treatment of the CFIDS condition, which is so personally sensitive. It is also important to distinguish the square waveform stimulator from a “T.E.N.S.” type apparatus, and the “twitch” mode from the “tetanus” mode. The T.E.N.S. stimulator, and “tetanus” mode of the Sparkie stimulator, will not work for the applications described herein. Neither, are the prior art procedures the same (in any mode) as that of the invention.

Generally, the prescribed or utilized techniques suggested and taught by this invention, are unique to stimulator technology. The stimulation of this invention is targeted at muscle “trigger points” and at the inter-vertebral spaces along the spine, and the nerve plexuses. Stimulation is applied via fixed electrical probes to the soft inter-vertebral spaces on either side of the spine of each individual with back pain, for example. The general treatment regimen starts at the base of the skull, and proceeds successively downward to the sacrum at the lower tip of the spine. In order to work properly, the patient needs to feel the stimulus. The parts of the body have different stimulus sensitivities. For example, when applying the stimulating current to the head of a patient; most patients will feel the stimulus at a 1-milliampere level. Applying the stimulus to the back of the same individual, the milliamps would likely have to be increased towards the upper amperage range of about 33 milliamps. Individuals, themselves, vary in sensitivity, as well as the different sensitivity levels associated with the different parts of the body. Placement of the electrodes also depends on the condition being treated. For example, for a stroke patient, or a patient with nerve damage, carpal tunnel syndrome, and victims of assault, i.e. low relationship to threshold and action potential, the stimulation site is the large nerve plexus. For high action potential conditions and diseases, such as fibromyalgia, psoriasis, etc., the action sites are local, and comprise trigger point sites.

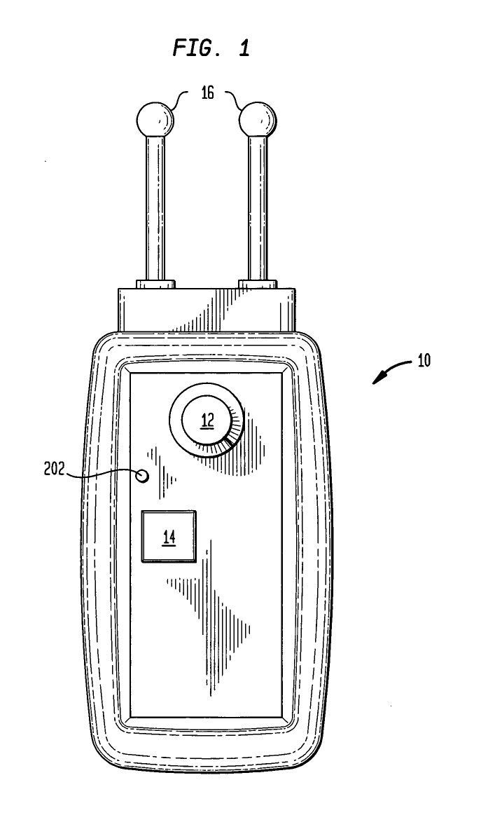

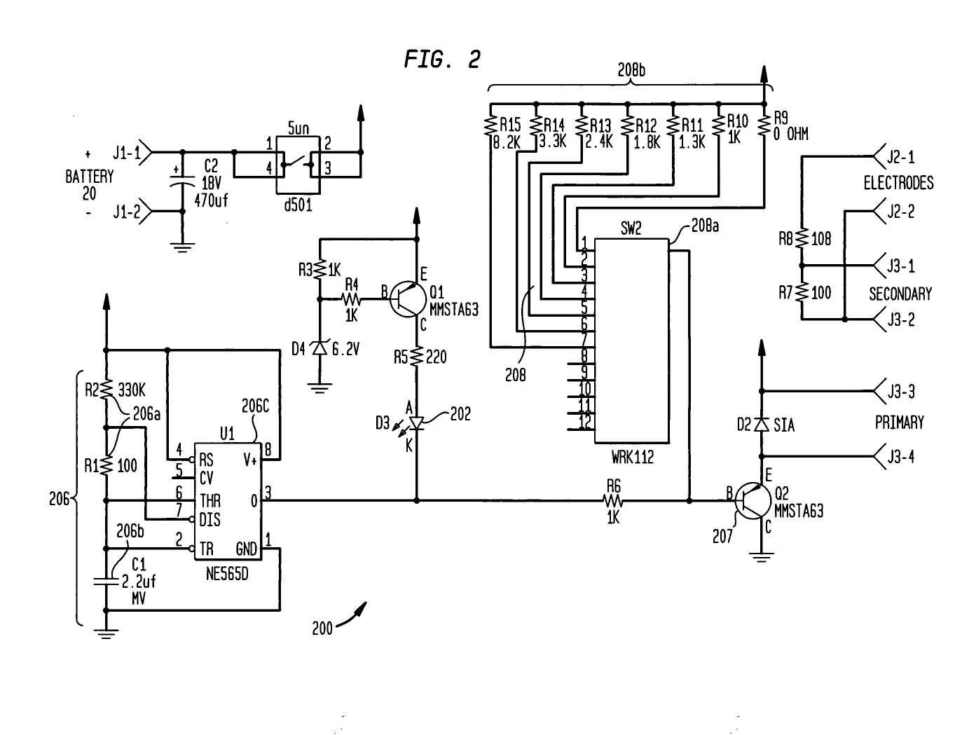

Now referring to FIG. 1, the handheld stimulator 10 of this invention, is illustrated. The stimulator 10 is similar to the Sparkie simulator, with at least one important difference. It comprises a potentiometer or rheostat 12, for adjusting the current of the electrical pulses, in order to find a comfort and effectiveness level for each individual being treated. The pulsing, or periodic electrical stimulation is designed to change the neurogenic character of the nerves. The pulses are designed to cause the nerves to revert to a normal condition, i.e. there is a readjustment of the electrochemical anomalies in the nervous system of the individual at the trigger sites, or at the nerve nexus. The stimulator 10 of this invention provides a substantially square-type wave output signal at the end of the fixed probes 16. The handheld stimulator 10 also comprises a pressure-sensitive switch 14, and an indicator (LED) light 18, to signal that current is flowing through the electrodes 16. The handheld stimulator 10 is operative by placing electrodes 16 against the patient at a desired location. The pressure switch 14 is depressed for a limited time, and the potentiometer 12 is adjusted to the sensitivity level of the patient. The indicator 18 flashes, as the pulses are administered to the site. The handheld stimulator is powered by a nine-volt battery 20, as depicted in circuit 200 of FIG. 2, which provides 2-second pulses to the electrodes 16 via the activation switch 14.

Referring to FIG. 2, the circuit 200 of the handheld stimulator 10 of FIG. 1, is illustrated. A light emitting diode 202, indicates when the stimulator is in operation. Transitors 204, (MMBTA63 PNP Darlington transitors) amplify the signals. Two resistors 206a and 206b, respectively, and timer chip 206C (NE555D) convert the direct current of battery 20 into a substantial square wave. Other suitable circuit components can also be used for this purpose.

Current control circuit 208 includes switch 208a and a plurality of resistors 208b, respectively. Each of the plurality of resistors comprises a different level of resistance. Switch 208a comprises a rotary switch MRK112, which provides a potentiometer function to increase or decrease the current ultimately provided to electrodes 16.

Saw-tooth waves, as supplied by the Sparkie simulator in the tetanus mode, and the T.E.N.S. machine are ineffective in treating the many neurogenic diseases and conditions specified by the current invention.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. An electrical stimulator providing a neurogenic improvement to a body site, comprising means for providing a pulsed or periodic output of electrical signals of substantially square waveform, said electrical stimulator having adjustment means for adjusting amperage of said electrical signals in order to adjust said electrical signals to comfort and sensitivity levels of a patient, whereby effectiveness of treatment of an individual being treated is neurogenically improved.

2. The electrical stimulator in accordance with claim 1, wherein said means for providing said electrical signals provides a stream of approximately two pulses per second.

3. The apparatus in accordance with claim 1, wherein said adjustment means comprises means for adjusting said current output between approximately 1 and 33 milliamps.

4. The apparatus in accordance with claim 1, wherein said adjustment means comprises a potentiometer.

5. A method for treating neurogenic ailments, said method comprising the steps of:
(a) generating a substantially periodic output electrical signal for application to nerve sites of an individual in need of neurogenic improvement, said periodic output electrical signal comprising substantially a square wave; and
(b) Stimulating nerve sits of said individual with the output electrical signal of step (a), in order to improve a neurogenic condition or ailment of the individual.

6. A method for treating neurogenic ailments, said method comprising the steps of:
(a) generating a periodic output electrical signals for relieving pain in an individual, said periodic output electrical signals having an adjustable current level between approximately 1 and 33 milliamps and being substantially a square wave; and
(b) stimulating neurogenic sites of said individual with said electrical signals of step (a) in order to improve neurogenic ailments and conditions of said individual.

7. A method of treating neurogenic ailments and conditions comprising the steps of:
(a) generating pulsed, substantially square wave electrical signals;
(b) adjusting a current level of said pulsed, substantially square wave electrical signals of step (a) in order to adjust the signal to individual sensitivity; and
(c) stimulating neurogenic sites of said individual with said electrical signals in order to improve an ailment or condition of said individual.

8. An electrical stimulator providing a neurogenic improvement to a body site, comprising means for providing a pulsed or periodic output of electrical signals of substantially square waveform, said electrical stimulator having adjustment means for adjusting amperage of said electrical signals in order to adjust said electrical signals to comfort and sensitivity levels of a patient, and a pair of fixed electrodes to administer said pulsed, or periodic output, whereby effectiveness of treatment of an individual being treated is neurogenically improved.

9. The electrical stimulator in accordance with claim 8, wherein said means for providing said electrical signals provides a stream of approximately two pulses per second.

10. The apparatus in accordance with claim 8, wherein said adjustment means comprises means for adjusting said current output between approximately 1 and 33 milliamps.

11. The apparatus in accordance with claim 8, wherein said adjustment means comprises means for adjusting said current output comprises a potentiometer.
